(19) United States
(12) Patent Application Publication
(10) Pub. No.: US 2017/0278420 A1
(43) Pub. Date: Sep. 28, 2017

(54) METHOD TO MONITOR IMPACTS ON THE HUMAN BODY WHILE PRACTICING RUNNING OR SPORTS

(71) Applicant: SAMSUNG ELETRÔNICA DA AMAZÔNIA LTDA., Campinas (BR)

(72) Inventors: Thiago JOÃO VIOL VIEIRA, Campinas (BR); Mateus LEME PAZINI, Campinas (BR)

(73) Assignee: SAMSUNG ELETRÔNICA DA AMAZÔNIA LTDA., Campinas (BR)

(21) Appl. No.: 15/366,625

(22) Filed: Dec. 1, 2016

(30) Foreign Application Priority Data
Mar. 23, 2016 (BR) 10 2016 006380 9

(51) Int. Cl. G09B 19/00; A61B 5/00; A63F 13/816; A63B 24/00; A63F 13/212; A63B 69/00; G01S 19/19; A63B 71/06

(52) U.S. Cl. CPC G09B 19/0038; G01S 19/19; A61B 5/4504; A63B 71/0622; A63B 24/0062; A63F 13/212; A63B 69/0028; A63F 13/816; A61B 2503/10; A61B 2562/0219; A63B 2220/53; A63B 2220/40; A63B 2071/0625

(57) ABSTRACT

A method to monitor impacts on the human body while practicing running or sports including: reading threshold values from personal information provided by the user; reading from a server the updated threshold values for training impact zones and reading the daily accumulated goal previously defined; reading data from an accelerometer sensor of a mobile device attached to the human body such as smartphones or wearable devices; calculating acceleration from data read from the accelerometer sensor; converting the measured acceleration into body weight units; obtaining date and time information, and optionally, satellite tracking data; checking if user has exceeded the appropriate level of impact conditions acceptable according to the training impact zones; notifying the user if he has exceeded appropriate level of impact conditions when achieved the beneficial goal of daily cumulative impact through an alarm; and displaying results/statistics of the training to the user.

### FIELD OF THE INVENTION

The present invention refers to a method to measure and monitor the impacts on the human body in physical or sports activities, specially running, through sensors in mobile devices such as smartphones and wearable devices (such as smartwatches, fitness bands and similar) aiming to determine whether the impact can be harmful to user's body and/or, in some cases, ensuring a minimum healthful amount of impact on the bones which can prevent some diseases such as osteoporosis.

### BACKGROUND OF THE INVENTION

Running (or jogging) is one of the most popular exercises and there are multiple benefits in doing such physical activity. Regular running builds strong bones, improves cardiovascular fitness and helps to maintain a healthy weight.

Bone density (or bone mineral density—BMD) is a medical term normally referring to the amount of mineral matter per square centimeter of the bones. Bone mineral density is used in clinical medicine as an indirect indicator of osteoporosis and fracture risk. There is a statistical association between poor bone densities and higher probability of fracture.

Studies in medical literature revealed that stress fractures are more than 10% of all running sportspersons lesions. Stress fractures are microscopic fissures on bones caused by a sum of impact quantities. Among the causes of stress fractures are: sudden increase of training intensity, volume or kind of training, which is affected by factors as type of shoes, type of terrain and type of stride.

However, it is critical to understand that the amount of impact on training like running can be healthy or harmful according to each kind of person.

Most common lesions related to running are reported on feet, ankles, knees, hips, column and even head. Consequently, it is important to sportspersons to evaluate how much impact is transmitted to their bodies while practicing sports or physical activities.

The impact force depends on factors like the body mass, the speed that feet hit the ground, the ground material and the way the impact force is absorbed. The ground reaction force (Ground Reaction Force—GRF) upon impact “is considered to be the most basic element which causes running related injuries”, as depicted on FIG. 5.

As each person responds individually to impacts, the impacts that may cause injury to one person may not be as severe to another. Some factors to be considered while measuring impacts on an individual are described as follows:
Gender: Females have more chance to have stress fractures than males (due to alimentary disorder, lack of menstrual cycle and higher probability to develop osteoporosis than males).
Age: Human bones reach their max strength around age of 30 to 35 years. The risk of stress fractures is higher for females with more than 65 years and males with more than 70 years. There is a higher probability of osteoporosis development for females with more than 50 years.
Weight: the more a person weights, the higher is the impact force (according to Newton's 2nd Law of motion: Force=Mass×Acceleration).
Duration: the more the impact lasts the more it can be harmful to a person due to cumulative effect.
Acceleration: higher levels of acceleration results in higher impact force on human body (again: Force=Mass×Acceleration).
Body Location: which part of the body in which the mobile device is attached to measure the impact.

The present invention relates to the following technologies, solutions and publications:

The accelerometer measures the physical acceleration, which is the acceleration it experiences relative to freefall and the acceleration felt by people and objects. In other words, at any point in space of time the equivalence principle guarantees the existence of a local inertial frame, and an accelerometer measures the acceleration relative to that frame. Such accelerations are popularly measured in terms of g-force.

An accelerometer at rest relative to the Earth's surface will indicate approximately 1 g upwards, because any point on the Earth's surface is accelerating upwards relative to the local inertial frame (the frame of a freely falling object near the surface). To obtain the acceleration due to motion with respect to the Earth, this “gravity offset” must be subtracted and corrections made for effects caused by the Earth's rotation relative to the inertial frame. The reason for the appearance of a gravitational offset is Einstein's equivalence principle, which states that the effects of gravity on an object are indistinguishable from acceleration.

For the practical purposes of finding the acceleration of objects with respect to the Earth, such as for use in an inertial navigation system, knowledge of local gravity is required. It can be obtained either by calibrating the device at rest, or from a known model of gravity at the approximate current position.

Conceptually, an accelerometer behaves as a damped mass on a spring. When the accelerometer experiences acceleration, the mass is displaced to the point that the spring is able to accelerate the mass at the same rate as the casing. The displacement is then measured to provide the acceleration.

Modern accelerometers are often small micro-electromechanical systems (MEMS), and are indeed the simplest MEMS devices as possible, consisting of little more than a cantilever beam with a proof mass (also known as seismic mass). Damping results from the residual gas sealed in the device. As long as the Q-factor is not too low, damping does not result in a lower sensitivity. The present invention makes use of accelerometer sensor in order to detect and measure the amount of impact received.

Body Weight (BW) are body weight exercises, also known as “calisthenics” in Brazil is a kind of training that uses the own body weight or everyday utensils. It does not require free weights and machines with load.

The application “S-Health”, by Samsung, consists in an all-in-one companion for a user's healthy lifestyle. With the S-Health application, the user can track his every day's activities, get coaching to reach his daily goals, and improve his fitness with various training programs, measure and understand his health with trackers such as heart rates and SpO₂ that use the built-in sensors in devices and smartwatches available in market.

The present invention proposes functionalities analogous to the pedometer feature (which accumulates the number of steps in a period) and to the heart rate monitor (which keeps the heart frequency within a healthy range), while monitoring the impact on the human body while practicing physical or sports activities, and warns the user in case the maximum allowed impact level is reached.

The dissertation “Measurement of Bone Exercise”, by Riikka Ahola (Faculty of Medicine of the University of Oulu, 2010), proposes a Daily Impact Score to describe individual daily osteogenic loading based on controlled training. It was intended to reveal the determinants of physical activity or exercise beneficial for the bone, using novel accelerometer-based measurement of bone loading, the paper indicated that the tested accelerometer-based method can be used to measure impacts generated in daily physical activities and bone exercise.

According to the article “Effects of High-Impact Training on Bone and Articular Cartilage: 12-Month Randomized Controlled Quantitative MRI Study” (Journal of Bone and Mineral Research, 2014), the simultaneous effect of bone-favorable high-impact training on these diseases is not well understood and it is a controversial issue. This paper has evaluated the effects of high-impact exercise on bone mineral content (BMC) and the estimated biochemical composition of knee cartilage in postmenopausal women with mild knee osteoarthritis. Progressively implemented high-impact training, which increased bone mass, did not affect the biochemical composition of cartilage and may be feasible in the prevention of osteoporosis and physical performance-related risk factors.

### DESCRIPTION OF THE RELATED ART

The patent document US 2015/040685 A1, titled “Impact Sensing, Evaluation & Tracking System”, published on Feb. 12, 2015, by Headcase, proposes an impact sensing system, evaluation and tracking, measuring the impact on the head of athletes who use helmets or other headwear while practicing sports, helping to assess the severity of impact and detecting the risk concussion and warning medical care issues using a computerized assessment. The present invention differs from document US 2015/040685 A1 because it monitors vertical impacts on the human body (feet, ankles, knees, hips, spine and not just head), while practicing physical or sports activities such as walking, running, dancing and jumping, measure and monitor the level of impact that your body is suffering over time, determines whether the impact can be harmful to your body. Further, the present invention uses an impact assessment scoring system (represented by colors), the threshold values may be imported from a server, in order to keep it updated with advances in sportive medicine field, the system provides useful information to users to avoid injuries and help people with diseases such as osteoporosis.

The patent document U.S. Pat. No. 8,333,104 B2, titled “Measuring Instrument for the Detection and Evaluation of an Impact”, published on Dec. 18, 2012, by Austrian Res Centers, Oberleitner Andreas and Austrian Institute of Technology, proposes a measuring instrument for detecting and evaluating an impact or shock of a collision. The present invention differs from document U.S. Pat. No. 8,333,104 B2 because it monitors the vertical impacts on the human body (feet, ankles, knees, hips, head and spine), through sensors of a mobile device (smartphones and wearable devices), while practicing physical or sports activities such as walking, running, dancing and jumping, measure and monitor the level of impact that your body is suffering over time, provides useful information to users to avoid injuries and to help people with diseases such as osteoporosis.

The patent document US 2009/000377 A1 titled “Brain Impact Measurement System”, published on Jan. 1, 2009, by Shipps Clay, Andic Hikmet and Bonfeld Jesse, proposes an impact measurement system in the brain and skull using a device that includes an accelerometer triaxial. The present invention differs from document US 2009/000377 A1 because it monitors impacts on the human body (feet, ankles, knees, hips, spine and not only of the brain and cranium), through sensors of a mobile device (smartphones and wearable devices) not being limited to the accelerometer, while practicing physical or sports activities such as walking, running, dancing and jumping, measure and monitor the level of impact that your body is suffering over time, determines whether the impact can be harmful to your body; using an impact assessment scoring system (represented by colors), the updated threshold values may be imported from a server, in order to keep it updated with advances in sportive medicine field, the system provides useful information to users to avoid injuries and to help people with diseases such as osteoporosis.

The patent document CN 203634171 titled “Exercise Load Measuring Shoe”, by Wang Xihua, Wu Jianchun and Yang Bo, published on Jun. 11, 2014, is a utility model that proposes to measure the load exercises using a device in a sock in the shoe. The present invention differs from document CN 203634171 because it monitors impacts on the human body while practicing physical or sports activities not being limited to feet; through a sensors of a mobile device (smartphones and wearable devices), in order to measure and monitor the level of impact that your body is suffering over time and determine if this impact can be harmful to your body.

The patent document CN 103637805 titled “Shoes and Method for Measuring Exercise Load”, by Wang Xihua, Wu Jianchun and Yang Bo, published on Mar. 19, 2014, proposes a method for quantitatively measuring the exercise load using a device attached to the shoes and determine the proper and healthy exercise for each person. The present invention differs from document CN 103637805 because it monitors impacts on the human body while practicing physical or sports activities; through sensors of a mobile device (smartphones and wearable devices), in order to measure and monitor the level of impact that human body is suffering over time, determine if this impact can be harmful to human body using an impact assessment scoring system (represented by colors), with the difference that updated threshold values for men and women can be imported from a server (cloud) in order to keep it up to date with advances in sports medicine field, the system provides useful information for users to avoid injuries.

The application “Linx Impact Assessment System”, by Linx IAS, proposes the automatic logging and storing of the head impacts in sports and activities, using an impact assessment system score and makes some decisions. The present invention differs from application because it monitors impacts on different parts of the human body (feet, ankles, knees, hips, spine, beyond the head), while practicing physical or sports activities such as walking, running, dancing and jumping, through a sensor device (smartphones and wearable devices), measure and monitor the level of impact that human body is suffering over time, determines whether the impact can be harmful to human body; using an impact assessment scoring system (represented by colors), where the scoring system is not fixed and it adapts with external data (cloud) in order to keep it up to date with the advances in the sports medical field, provides useful information to users to avoid injuries and to help people with diseases such as osteoporosis. The Linx application, differently, is normally attached to the user's helmet and monitors impacts directly on the user's head in critical situations such as in the war battlefield, special missions and sports with contact, such as rugby, American football or box.

### SUMMARY OF THE INVENTION

The present invention presents a method to monitor the impacts transmitted to the body by using sensors in a mobile device while practicing physical activities or sports activities such as walking, running, dancing, skipping, and provides useful information to users to avoid injuries related to impact forces.

The present invention uses sensors built in mobile devices to determine whether such impacts can be harmful to user's body and/or, in some cases, ensuring a minimum healthful amount of impact on the bones which can prevent some diseases such as osteoporosis. The main objectives achieved by the present invention consists on:
Monitor the impact on user's body and to provide information to the user about the received impact allowing the user to determine whether it is harmful or not, taking into account the limits per exercise duration, gender, age and location of attachment. The way the information is provided to user is similar to the heart rate monitor feature, which monitors user's heart rate in order to not exceed a predefined desired limit. This concept is analogous to the “fitness zone” monitored by a HRM (Heart-Rate Monitor), as depicted in FIG. 9.
Monitor the cumulative impact within a given period of time and checks if a desired accumulated amount of impact has been reached. This concept is similar to the pedometer feature that cumulatively counts steps within a given period of time against a predefined daily steps goal, as depicted in FIG. 10.

The impact monitor feature is an impact assessment scoring system that is represented by colors and labels according to the impact training zones (e.g. safe, caution, danger).

It is considered high the probability of use of the present invention by athletes, coaches, personal trainers, health clubs, physicians, patients and other users while they are walking, running, jumping and dancing. The benefits are even greater if the proposed concepts are integrated into existing platforms, such as S-Health by Samsung for fitness and healthy, as many others.

The objectives and advantages of the present invention are achieved through a method to monitor impacts on the user's body, comprising the steps of:
reading threshold values from information provided by the user;
reading from a server the threshold values for training zones and the daily impact goals according to the information inserted by the user;
reading data from an accelerometer sensor of a mobile device attached to the human body;
calculating the acceleration vector from the data read from the accelerometer sensor;
converting the acceleration into body weight (BW) units;
obtaining date, time and location data from satellite (GPS);
checking if user has exceeded the suitable conditions of acceptable level of impact; and
notifying the user whether he has exceeded the suitable conditions for the level of impact through an alarm vibrate, an audible sound and/or visual alarm); and
displaying to the user the results/statistics about the training.

The present invention takes advantage of a mobile device containing multiple axis accelerometer hardware to measure the intensity of the impact force for fitness and health purposes. Accelerometer sensors can detect acceleration (movement) and its amount (measure). Such data can be recorded and processed in order to calculate the impact being detected by the mobile device (hence to the person carrying or wearing it during the training).

The invention considers in the calculation of the impact the location where device is attachment and it estimates the amount of impact which is healthy or not according to gender and age. The updated values for thresholds used to determine the training zones can be read from a remote entity such as a server. Color codes can be used to easily inform to user about the training zones.

With such information it is possible to warn users about the level of impact on his body and determine if that impact is healthy or not. Based on this, users can take the appropriate actions to minimize injuries like per example changing the training terrain, changing the type of shoes, reducing the training speed, stepping on the floor in a more appropriate way, and so on:
Lower levels of impact can prevent pain in feet, ankles, knees, hips and column (bones and joints diseases). This is analogous of how a heart rate monitor (HRM) monitors the exercise effort within a predefined heart rate working zone.
The adequate impact can help people in risk to develop osteoporosis or other bone-related disease to stimulate bone mass due to the pressure applied to the bones. This is analogous of how a pedometer monitors whether a predefined daily step goal has accumulatively been reached.

### BRIEF DESCRIPTION OF THE DRAWINGS

The objectives and advantages of the present invention will become clearer through the following detailed description of an example but non-limitative embodiment of the invention, in view of the attached drawings, wherein:

FIG. 1 depicts the human body regions where device with accelerometer can be attached (lower limb, center, upper limb).

FIG. 2 depicts the relationship of Impact Training Zones along a geographical location using a color code system.

FIG. 3 depicts the relationship of Impact Training Zones along the time using a color code system.

FIG. 4 shows the difference between isolated axis acceleration and the total acceleration.

FIG. 5 depicts the typical vertical ground reaction force for running and walking activities.

FIG. 6 depicts the peak force of a stride which is the force region target for impact calculation on this method.

FIG. 7 depicts the Acceleration peak for 10 milliseconds samples.

FIG. 8 depicts the Impact monitor flowchart.

FIG. 9 depicts a Graphical User Interface (GUI) exemplifying the last training info average impact in analogy to the HRM (Heart-Rate Monitor) from S-Health application.

FIG. 10 depicts a Graphical User Interface (GUI) exemplifying the cumulative impact and what is desirable to achieve the goal in analogy to the Pedometer feature from S-Health application.

### DETAILED DESCRIPTION OF THE INVENTION

The present invention monitors impacts transferred to the human body while doing physical or sports activities such walking, running, dancing, skipping and to provide useful information to users avoid injuries related to impact forces.

The present invention proposes to use a mobile device that has multiple axis accelerometer hardware to measure the intensity of the impact force for fitness or health purposes. Accelerometer sensors can detect/measure acceleration direction (axis x, y, z) and intensity. Such data can be recorded and processed in order to calculate the shock being detected by the device (hence to the person carrying or wearing it during the training).

With such information it is possible to warn users about the level of impact on his body and to determine if it is healthy or not to him/her. Based on this, users can take appropriate actions to minimize injuries like for example changing the training ground, changing the type of shoes, reducing training speed or stepping on the floor in a more appropriate way.

Based on some user inputs such as gender, weight, region of the mobile device in the human body, target level of impact training; it is possible to determine secure levels of impact (e.g. safe/caution/danger zones).

In addition to user provided data, the device can automatically detect parameters like duration, acceleration, geographic coordinates, date, time and combine them to suggest to user training zones and training programs.

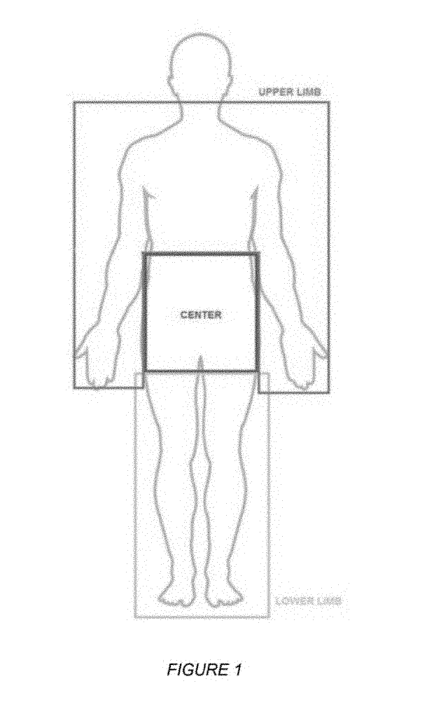

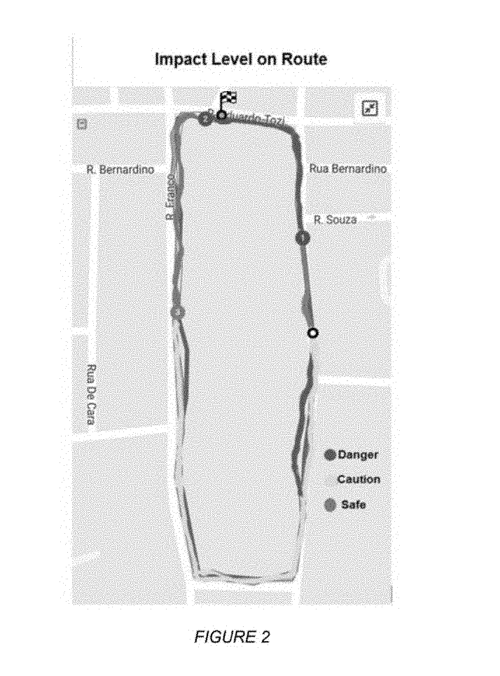

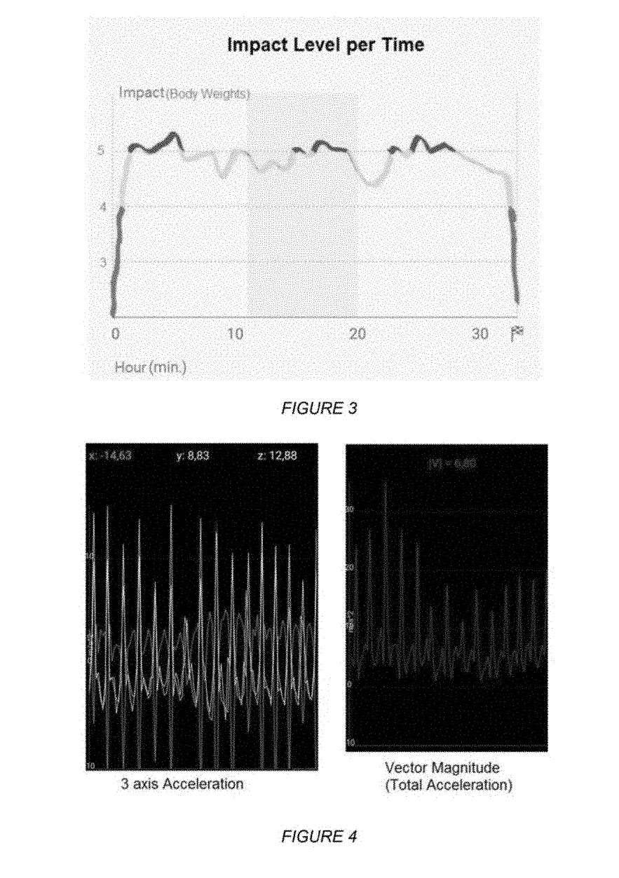

Proposed Invention features:
Definition of thresholds (values) for impact force intensity and categorize them in levels (safe/caution/danger).
Customization of Impact Training Zones thresholds according to:
Region of the body where device is attached (info provide by user) and consult impact parameters in server, as shown in FIG. 1.
Gender (info provide by user).
Age (info provide by user).
Identification of average intensity based on number of samples per period of time (to avoid single peaks interference on final results).
User alert in case a danger Impact Training Zone is active for a given period of time (vibrate/audio/visual alarm) during the training.
Indication if amount of required impact per day has been reached (cumulative daily impact to prevent osteoporosis).
Association of geographical coordinates (GPS) to identify levels of impact per route sections. See Picture 2.
Graphical plotting correlating speed, altitude, time, impact training zone info, as shown in FIG. 3.
Association of amount of time spent on a given level of impact.
Inclusion of gamification rules offering awards to user (like badges per example in case Impact Training Zone training was achieved for a given amount of time and sharing of user results to compare to other users in a ranking).
Ability to provide hints automatically to user on how to reduce impacts in case danger Impact Training Zone alert was triggered (suggest modifications on foot stride, ground terrain, shoes, reduce weight, increase step rate at a given speed).
Data logging capabilities: be integrated with health/well-being platforms.

### Impact Training Zones:

Linear Acceleration is the force along an axis (x, y or z) excluding earth's gravity. The three components of motion for an individual (and their related axes) are forward (roll, x), vertical (yaw, y), and side (pitch, z). Linear acceleration is measured in m/s². Using device's linear acceleration sensor provides a three-dimensional vector representing acceleration along each device axis, excluding gravity, as shown in FIG. 4.

Method obtains the three axis readings and determines the magnitude of the sensor reading using standard vector math. This effectively takes into account the energy from all three axes simultaneously. Vector Magnitude in G acceleration=square root (X*X+Y*Y+Z*Z).

According to Newton's law, Force=Mass×Acceleration. The present invention is related to measure the force of an impact detected by the device. The bigger is the mass (person's weight) the bigger is the resulting impact. The same is true for acceleration.

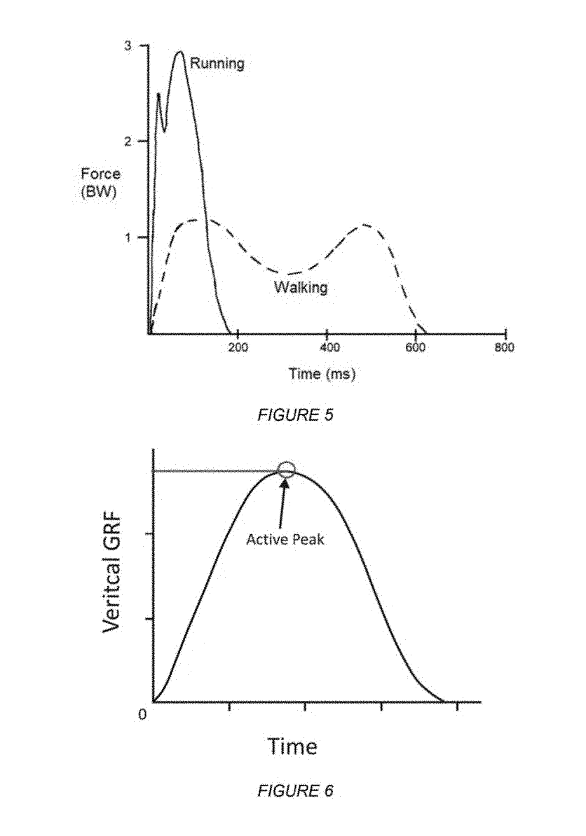

So, it is supposed that when a 70 Kg weight's person is running and stride the ground with an acceleration of 2 times the gravity which is ˜20 m/s², then the Ground Reaction Force (GRF—shown in FIG. 5) will be according to function “Force=mass times acceleration”:

F=m×a;

F=70×20;

F=1400 N//where N=Newtons.

Associating body weight (BW) with impact:

Converting Mass into Force, a 70 Kg person will weight: 70*10=700 N. //assuming earth's gravity is 10 m/s².

So an impact of 1400 N represents 1400/700=2 times the body weight of a 70 Kg mass person in Earth.

So there is a direct relation of the applied “g” force with body weight.

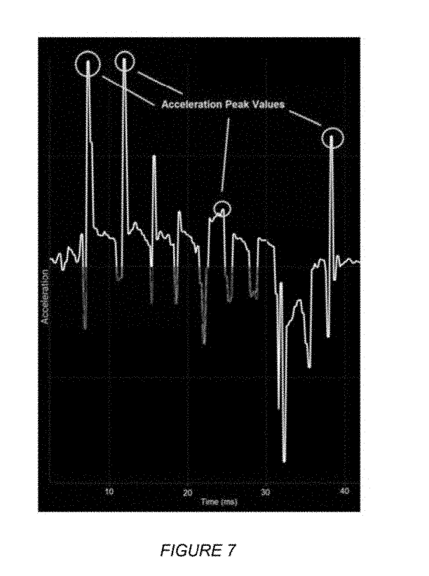

The force of an impact peak (shown in FIG. 6) can have different consequences according to each person. So the present invention relies on scientific data to propose different levels to represent impact intensity (called impact training zones) in easier way. An example of impact training zones color code is indicated below:
Green Zone: Safe. Represents impacts lower than yellow zone and that can be considered safe to user's practice his/her exercise.
Yellow Zone: Caution. Represents a range of impact higher than green zone but lower than red zone which is determined by a level of impact that may injure user's body so caution on training is required.
Red Zone: Danger. Represents a range of impact higher than yellow zone that statistically can cause injuries to user's body while practicing exercise.

According to Clark (National Academy of Sports Medicine, 2002) and Peter Merton McGinni (Biomechanics of Sport and Exercise):
During Walking GRF=1-1.5 times body weight.
During Running GRF=2-5 times body weight.
During Jumping GRF=4-11 times body weight.

The present invention proposes the impact training zones values relying in conventional approximation parameters indicated below (for males):
Green (male): up to 4 times body weight.
Yellow (male): from 4 to 5 times body weight.
Red (male): higher than 5 times body weight.

### Adjusting Impact Training Zones Per Gender:

The female skeleton is generally less massive, smoother, and more delicate than the male. Males in general are seen to have denser, stronger bones, tendons, and ligaments.

Females in general have lower total muscle mass than males, and also having lower muscle mass in comparison to total body mass. Males convert more of their caloric intake into muscle and expendable circulating energy reserves, while females tend to convert more into fat deposits. As a consequence, males are generally physically stronger than females.

Gross measures of body strength suggest a 40-50% difference in upper body strength between the genders, and a 20-30% difference in lower body strength.

As there are gender-related differences in human physiology it is relevant this method to consider these facts introducing a weighted value for the impact training zones for women to prevent injuries in a more accurate way. An adjustable factor of 20% is introduced specifically for females as indicated below:
Green (female): up to 3.33 times body weight.
Yellow (female): from 3.33 to 4.16 times body weight.
Red (female): higher than 4.16 times body weight.

### Adjusting Impact Training Zones Per Site of Device Attachment:

Another factor that is critical to have better accuracy when calculating impact training zones is to understand the location of the body where the device is attached. Each region of human body suffers a different level of ground reaction force.

Acceleration attenuates as the shock wave propagates up the body. The closer the accelerometer is to the ground, i.e. the site of impact, the higher the acceleration values.

The signal is attenuated by about 50% between the lower limb and the head, and attenuation is present even in the ankle joint. Impact energy is absorbed by the whole locomotion system: the muscles, bone, ligaments and tendons.

Similarly, studies have reported a linear correlation coefficient of 0.90 between vertical peak GRF and tibial acceleration and 0.73 between peak GRF and waist acceleration.

This method defines 3 different regions of human body where device could be attached as well respective adjustment factors to consider acceleration attenuation across the body:
Lower limb (foot, ankle, knee)—contribution of 100%;
Center (waist, hips)—contribution of 70%;
Upper limb (arms, chest)—contribution of 50%.

In summary:

The parameters values (adjustment factors for gender and site of device attachment) for each impact training zone can be defined in a static way (hard coded in a software program) or they can be read by the device from an external source such as a server. This approach makes feasible to the device update such values to more accurate ones after their launch. It is important since currently there is a lack of precision on such values in biomechanics scientific literature. So once more research/studies in such field become available at scientific community the values can be adjusted.

The relevant acceleration measurements for this method are the ones that contain information of ground impact. Therefore, this method relies on peak values for accelerometer measurements only, discarding ascending/descending legs movement measurements.

In case peak acceleration is above the “Danger” impact training zone for a given period of time then the user is warned (vibrate/audio/visual alarm) to avoid injuries during the training session.

Again, such values above mentioned can be dynamically adjusted based on readings from a server, as shown in FIGS. 5 and 7.

It is noted that the invention is not limited to a specific number of levels of impacts and to the mentioned color codes. Three levels of impacts and the green, yellow and red colors were suggested to illustrate the functionality.

### Osteoporosis Prevention for Elderly Women:

Bone size and mass increase dramatically during growth, and peak bone mass is generally obtained around 10 years after skeletal growth has stopped. After this, bone mass slowly starts to decline. In women, there is rapid bone loss after menopause (after 50 years old) due to decreasing hormonal levels, especially estrogen.

The method performs the counting of the impacts above 4 BW until reach 60 on the same day, such that when overcoming this reference value, an audible alarm will be triggered each time there is a greater impact than 4 BW, so the person/women will know if you are doing an exercise that stimulates your bone formation. For example, step exercise where the person goes up and down on a platform, when you reach the 60 impacts >4 BW, it will sound the alarm completion of the exercise. Again, these values can be read by the device from an external source such as a server to allow a better tunning.

As females are in more risk than males for developing osteoporosis the present invention proposes a method to help women from 50 to 65 years to prevent bone loss and also to maintain physical performance to avoid falls.

This can be achieved by monitoring cumulative impacts in a given amount of body weights. The impact can be quantified by recording the number and intensity of acceleration peaks (impacts).

The impacts were analyzed at four acceleration levels according to the multiples of acceleration of gravity (g): low (0.3-2.4 g), moderate (2.5-3.8 g), high (3.9-5.3 g), and very high (5.4-9.8 g), where g=9.81 m/s² and 0 g is equated to standing still.

### Daily Impact Score:

In summary:

### Flow of the Invention:

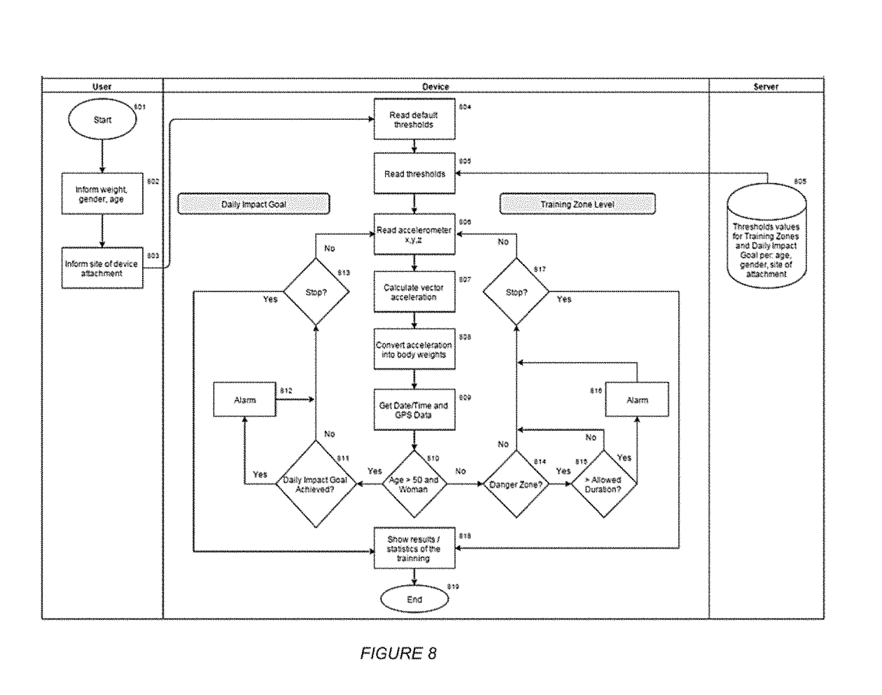

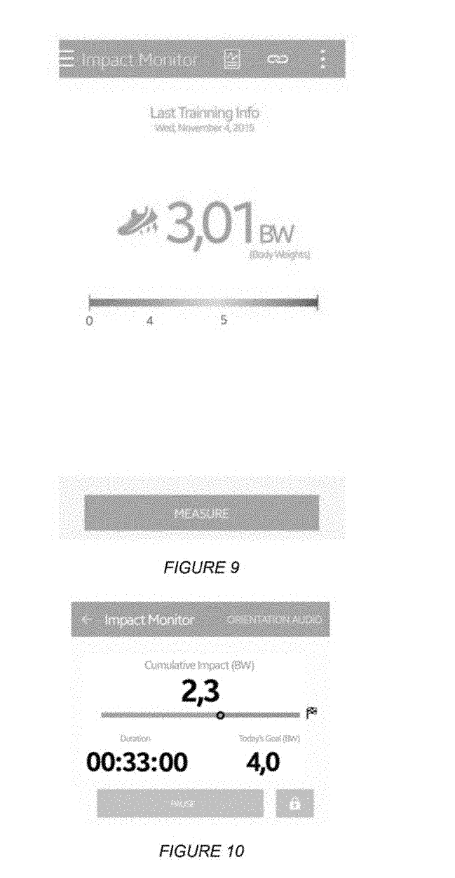

The flowchart of the invention comprises the following steps as shown in FIG. 8.

User: The user starts the feature sports impact monitor (801) on a platform of e-Health at the mobile device (smartphones or wearable devices), informing the personal data such as weight, gender, age or the application extracting the data already stored (802) and, at last, the user informs the contact-point of mobile device attachment in your body (803).

Server: The threshold values for each variable in training zones and daily impact goal can be read/import automatically by the mobile device from an external server or “cloud” (805).

Device: The mobile device reads (804) the default threshold values for each variable and also reads (805) threshold values of each variable of training zones and daily impact goal on an external server or “cloud” and it adjusts dynamically using the updated parameters.

The accelerometer sensor detects and reads the coordinates x, y, z (806), so the acceleration vector is calculated (807), and the acceleration is converted to body weights (808) and automatically obtained and the date, time, GPS data and other parameters (809).

Based on the reading of the data, the method checks whether the user is a woman and has age more 50 years (810):
If positive, the daily impact goal is accessed (left), if the goal of the daily impact has been achieved—higher than 4 BW (811), an audible alarm sounds (812) to avoid injury and complete the exercise, and the next step; the application asks if you want to stops the training (813), if yes; the application displays the results and statistics training (818). If no, the mobile device again starts reading the coordinates by the accelerometer sensor (806).
If negative, the training zone level is accessed (right), if the impact level is Danger (814) with a greater estimated time (815), an audible alarm sounds (816) to avoid injury, and the next step; the application asks if you want to stop (817). In positive case the application displays the results and statistics training (818). In negative case the mobile device again starts reading the coordinates by the accelerometer sensor (806).

In the end of the impact monitoring (819), application displays the results and statistics of the training (818).

Although the present invention has been described in connection with certain preferred embodiment, it should be understood that it is not intended to limit the invention to that particular embodiment. Rather, it is intended to cover all alternatives, modifications and equivalents possible within the spirit and scope of the invention as defined by the appended claims.

1. A method to monitor the effects of an impact on the human body while practicing running or sports activities through a mobile device attached to the human body containing accelerometer sensor capable of measuring the intensity of impact forces on different parts of the human body and storing information on the device for further analysis, the method comprising of:
reading limits from information personal user input, including gender, age and weight;
reading from a server, the updated threshold values for training impact zones, such as safe, caution and danger;
reading the cumulative daily impact goal previously defined;
reading data from an accelerometer sensor of the mobile device attached to the human body, such as smart phones or wearable devices;
calculating the acceleration vector from data read from the accelerometer sensor;
converting the acceleration in body weight units (BW);
obtaining date and time information from the system, and optionally, the satellite tracking data;
checking if user has exceeded appropriate conditions of acceptable level of impact, according to the training impact zones;
assessing whether the level of impact is healthy through the following:
notifying the user if he has exceeded appropriate conditions of the level of impacts through an alarm;
notifying the user as soon as he has reached the beneficial goal of daily cumulative impact through an alarm; and
displaying training results/statistics to the user.

2. The method to monitor effects of an impact on the human body according to claim 1, further comprising defining different impact zones using variable values such as gender, age, duration, acceleration and body region where the device was attached.

3. The method to monitor effects of an impact on the human body according to claim 2, wherein the definition of the training impact zones is adjusted based on data obtained from external servers.

4. The method to monitor effects of an impact on the human body according to claim 1, wherein in response to evaluating the level of impact is healthy, suggesting to the user actions to minimize injuries such as changing the type of training ground, changing the type of shoes, reducing training speed and stepping on the floor in an appropriate way.

5. The method to monitor effects of an impact on the human body according to claim 1, wherein the geographic coordinates (GPS) are associated to levels of impact identified by path parts, by plotting graphics and reporting the detailed correlation between speed, altitude, time and level of impact information.

6. The method for monitoring effects of an impact on the human body according to claim 1, further including gamification approaches (gamification) such as award and scoring, related to achieved training goals associated with each impact zone.

7. The method to monitor effects of an impact on the human body according to claim 2, wherein the geographic coordinates (GPS) are associated to levels of impact identified by path parts, by plotting graphics and reporting the detailed correlation between speed, altitude, time and level of impact information.
